(19) United States
(12) Patent Application Publication
(10) Pub. No.: US 2026/0340868 A1
(43) Pub. Date: Oct. 29, 2026

(54) Method for Determining the Position of a Target Element in an Image of Interest of an Area of the Body

(71) Applicants: Université de Strasbourg, Strasbourg (FR); Hôpitaux Universitaires de Strasbourg (HUS), Strasbourg (FR); Fondation de Cooperation Scientifique, Strasbourg (FR)

(72) Inventors: Laurent GOFFIN, Strasbourg (FR); Lise LECOINTRE, Wantzenau (FR); Benoit GALLIX, Montpellier (FR)

(21) Appl. No.: 19/115,331

(22) PCT Filed: Sep. 27, 2023
(86) PCT No.: PCT/EP2023/076646

§ 371 Date: Mar. 26, 2025

(30) Foreign Application Priority Data
Sep. 28, 2022 (FR) FR2209860

(51) Int. Cl. G06T 7/73; A61B 5/00; G06T 7/00

(52) U.S. Cl. CPC G06T 7/74; A61B 5/418; A61B 5/489; G06T 7/0014; G06T 2207/30101; G06T 2207/30244

(57) ABSTRACT

interest of an area of the body The present invention relates to a method for determining the position of a target element in an image of interest (IM_{I}) of an area of the body, the area of the body comprising an intermediate element imaged on the image of interest (IM_{I}), the image of interest (IM_{I}) having been acquired by an imaging device (12), each image acquired by the imaging device (12) having a specific reference, referred to as the image reference (R_{I}), in which the elements of the image are referenced, the position of the imaging device (12) on each image acquisition being referenced in a fixed reference frame, referred to as the tracking reference frame (RS), the area of the body being stationary in the tracking reference frame (RS) during the implementation of the method.

### TECHNICAL FIELD OF THE INVENTION

The present invention relates to a method for determining the position of at least one target element on at least one image of interest of an area of the body. The present invention further relates to an associated determination system.

### BACKGROUND OF THE INVENTION

The therapeutic management of endometrial cancer depends on the estimation of the theoretical risk of recurrence and on lymph node metastasis. In practice, lymphadenectomy, resection of the lymph node chain, is performed only on patients at real risk of relapse. In order to reduce morbidity, the concept of sentinel node was developed to isolate the first node(s) draining a solid tumor.

The detection of the sentinel node is now based on a combined technique combining an injection of radioisotope before the intervention and either an injection of blue dye just after the anesthetic induction, or a colorimetric detection alone with indocyanine green intraoperatively.

Nevertheless, the failure rate of the detection, in particular a bilateral detection, of the sentinel node, is of about 25%. In addition, current procedures have several limitations, including reliability, accuracy, and potential complications related to the procedure.

Thereby, during key-hole surgery (also called laparoscopy), it is difficult to locate the sentinel node on key-hole surgery images.

### SUMMARY OF THE INVENTION

There is thus a need for a method and system to facilitate the detection of at least one target element, such as the sentinel node, on an image of an area of the body.

To this end, the subject matter of the present description is a method for determining the position of at least one target element on at least one image of interest of an area of the body, the area of the body comprising at least one intermediate element imaged on the image of interest, the image of interest having been acquired by an imaging device, each image acquired by the imaging device having a specific frame of reference, called the image frame of reference, wherein the position of the imaging device at each acquisition of images is identified in a fixed frame of reference, called the monitoring frame of reference, the area of the body being immobile in the monitoring frame of reference, the method being implemented by computer and comprising:
a. a preparation phase comprising the following steps:
   i. obtaining a geometrical representation, called local geometrical representation, of the intermediate element, the local geometrical representation being identified in a fixed geometrical frame of reference, called local frame of reference, the target element having a relative position with respect to the intermediate element which is known in the local frame of reference,
   ii. obtaining preparatory images of the area of the body, the preparatory images having been acquired by the imaging device for different positions of the imaging device, each position of the imaging device corresponding to a different angle of view of the area of the body,
   iii. The determination of a transformation, known as the local transformation, between the monitoring frame of reference and the local frame of reference according to the local geometric representation, the preparatory images, and the positions of the imaging device during the acquisition of the preparatory images,
b. an operating phase comprising the following steps:
   i. obtaining an image of interest of the area of the body,
   ii. the determination of a transformation, called an image transformation, between the local frame of reference and the image frame of the image of interest, the image transformation being determined depending upon the determined local transformation and the position of the imaging device during the acquisition of the image of interest, and
   iii. determination of the position of the target element on the image of interest according to the determined image transformation and to the known relative position of the target element relative to the intermediate element.

According to other particular embodiments, the method comprises one or more of the following features, taken individually or according to all technically possible combinations:
the steps of the operating phase are repeated over time for images of interest corresponding to different positions of the imaging device;
the exploitation phase includes a step of displaying the image of interest on a screen with a superimposed representation of the target element at the determined position on the image of interest;
the local geometric representation is obtained depending upon sectional images of the area of the body comprising the intermediate element and the target element, the sectional images having been acquired by a medical imaging device;
the step of determination of the local transformation comprising:
   a. the determination, for each preparatory image, of a geometrical representation, called an annotated representation, of the intermediate element, the annotated representations being identified in the monitoring frame of reference according to the position of the imaging device during the acquisition of said images,
   b. placing the local geometric representation at an initial position in the monitoring frame of reference,
   c. the determination, in the monitoring frame of reference, according to the annotated representations, of a representation, called a preparatory geometric representation, and
   d. the determination of the local transformation by registration of the local geometrical representation and the preparatory geometrical representation in the monitoring frame of reference (R_{S});
the preparatory geometrical representation is obtained by a projection of the annotated representations and an intersection of said projections;
the intermediate element has an invariant shape in projective geometry; and
the intermediate element is an arterial system, such as the iliac arterial system, and the target element is a lymph node, preferably a sentinel lymph node.

The present invention also relates to a system for determining the position of at least one target element on at least one image of interest of an area of the body, the area of the body comprising an intermediate element imaged on the image of interest, the image of interest having been acquired by an imaging device, each image acquired by the imaging device having a specific frame of reference, called the image frame of reference, wherein the image elements are identified, the position of the imaging device at each image acquisition being identified in a fixed frame of reference, called the monitoring frame of reference, configured to implement the steps of a method according to the invention; and
the system further comprises:
   a. the imaging device for the acquisition of preparatory images and of images of interest,
   b. a support on which the imaging device is mounted, and
   c. a monitoring device connected to the support for moving the imaging device and monitoring the movements of the imaging device.

The present description further relates to a computer program product on which is stored a computer program comprising program instructions, wherein the computer program being loaded on a data processing unit leads to the implementation of the method such as described hereinabove when the computer program is implemented on the data processing unit.

The present description further relates to a readable storage medium on which is stored a computer program comprising program instructions, the computer program being loaded on a data processing unit and leading to the implementation of a method such as described hereinabove when the computer program is implemented on the data processing unit.

The present description also relates to a surgical method comprising:
the placement of an imaging device in the patient's body (anesthetized),
the acquisition by the imaging device of preparatory images of an area of the body, the area of the body comprising an intermediate element (visible in the images) and a target element (either visible or not visible in the images),
the implementation of the preparatory phase of the determination method as described hereinabove depending upon the preparatory images acquired,
the acquisition by the imaging device of an image of interest of the area of the body, and
the implementation of the operating phase of the determination method as described above for the acquired image of interest.

### BRIEF DESCRIPTION OF THE DRAWINGS

Other features and advantages of the invention will appear upon reading hereinafter the description of the embodiments of the invention, given only as an example, and making reference to the following drawings:

FIG. 1, a schematic view of an example of a system for determining the position of a target element on images of interest of an area of the body,

FIG. 2, a flowchart of an example of implementation of a method for determining the position of a target element on images of interest of an area of the body, and

FIG. 3, a schematic view illustrating an example of obtaining a local geometric representation depending upon sectional images (coming from a medical imaging device),

FIG. 4, a schematic representation of an example of an annotated representation of an intermediate element, the annotated representation having been obtained from a preparatory image, and

FIG. 5, a schematic representation of an example of a registration of a local geometry and a preparatory geometry in the monitoring frame of reference.

### DETAILED DESCRIPTION OF SOME EMBODIMENTS

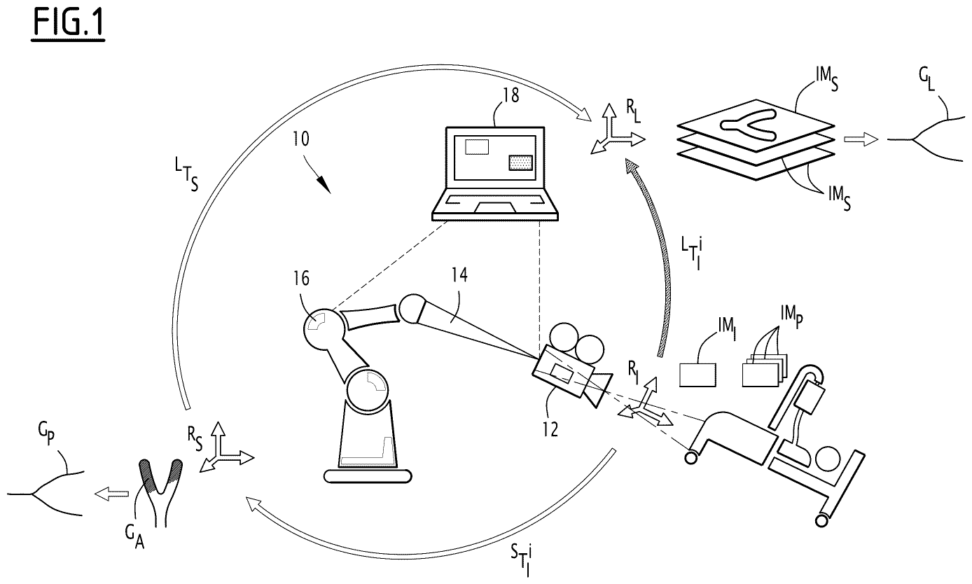

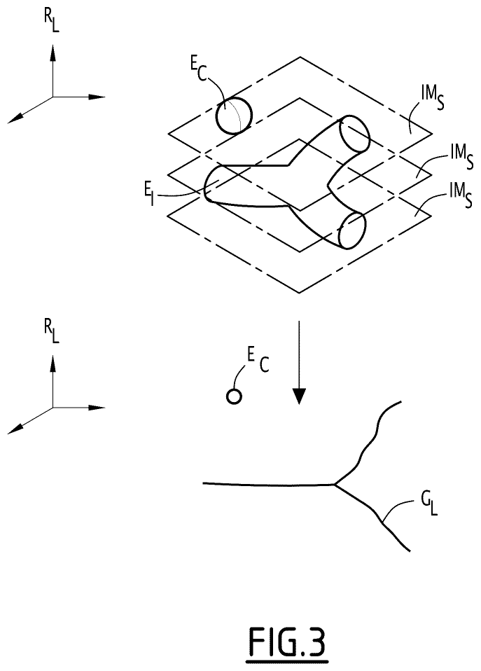

An example of a system 10 for determining the position of a target element E_{C} on images of interest of an area of the body Z is illustrated in FIG. 1. The target element E_{C} is illustrated in FIG. 3.

In the present example, the system 10 comprises an imaging device 12, a support 14, a monitoring device 16, and a calculator 18.

In a variant, the system 10 comprises only the calculator 18.

The imaging device 12 is suitable for acquiring images of an area of the body Z. The imaging device 12 is e.g. a camera.

In a main embodiment, the acquired images are two-dimensional (2D) images.

In a variant, the acquired images are three-dimensional images (3D), e.g. obtained directly from a 3D video stream or resulting from two 2D video streams combined to obtain the 3D images.

The support 14 supports the imaging device 12. The support 14 is suitable for being displaced, thereby allowing the displacement of the imaging device 12.

The support 14 is e.g. a tube into which the imaging device 12 is inserted.

The monitoring device 16 is connected to the support 14. The monitoring device 16 is suitable for moving the imaging device 12 and to follow the displacements of the support 14, and thus of the imaging device 12.

In one example, the monitoring device 16 is an instrumented arm.

The calculator 18 is e.g. connected by wired or wireless means (Wifi, Bluetooth), on the one hand, to the imaging device 12 so as to receive the images acquired by the imaging device 12, and, on the other hand, to the monitoring device 16 so as to obtain the position of the imaging device 12.

The calculator 18 is e.g. a computer in interaction with a computer program product.

The calculator 18 typically includes a processor comprising a data processing unit, memories and a data storage support reader. The calculator 18 also conventionally comprises a human-machine interface (keyboard), a screen (whether a touch-screen or not) and a mouse.

The computer program product includes a data storage medium.

The data storage support is a support readable by the calculator 18, usually by the data processing unit. The readable storage medium is a medium suitable for storing electronic instructions and apt to be coupled to a bus of a computer system.

As an example, the data storage support is a USB key, a diskette or a floppy disk, an optical disk, a CD-ROM, a magneto-optical disk, a ROM, a RAM, an EPROM, an EEPROM, a magnetic card or an optical card or else a perforated card.

The computer program containing program instructions is stored on the data storage medium.

The computer program can be loaded into the data processing unit calculator 18 and is suitable for leading to the implementation of a method for determining the position of a target element EC on images of interest of an area of the body Z.

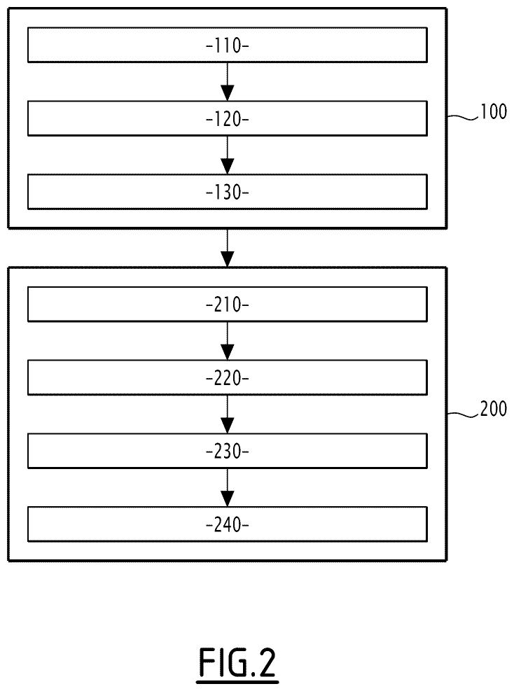

The operation of the device 10 will now be described with reference to the example shown in FIG. 2, which illustrates a flowchart of a method for determining the position of a target element E_{C} on images of interest of an area of the body Z, and FIGS. 1 and 3 to 5, which illustrate examples of the implementation of steps of the method.

The determination method described below is aimed at determining the position of a target element E_{C} on images of interest of an area of the body Z. The area of the body Z comprises at least one intermediate element E_{I} and at least one target element E_{C}. The intermediate element E_{I} is visible on the images of interest, the target element E_{C} is generally not visible on the images of interest (hidden by organs or tissues of the area of the body Z). It is understood that the intermediate element E_{I} and the target element E_{C} are close.

Preferably, the intermediate element E_{I} has an invariant shape in projective geometry, i.e. the shape of the intermediate element does not vary when the element is projected (e.g. in a plane). The intermediate element E_{I} has e.g. a tubular shape.

Preferably, the intermediate element E_{I} is an arterial system, such as the iliac arterial system, and the target element E_{C} is a lymph node, preferably a sentinel lymph node.

FIG. 1 illustrates in a general manner the geometrical frames of reference and transformations considered during the implementation of the present method.

More particularly, three distinct geometric frames of reference are considered, namely:
an image mark R_{I} (variable) specific to each image acquired by the imaging device 12 and in which the elements of the image are identified. The images considered thereafter in the description are the preparatory images IM_{P} and the images of interest IM_{I}.
a monitoring frame of reference R_{S} (fixed) wherein the positions of the imaging device 12 are identified during the acquisition of the different images. Thereby, the positions, in the monitoring frame of reference R_{S}, of the images acquired by the imaging device 12 depend on the position of the imaging device 12. The monitoring frame of reference R_{S} is e.g. a frame of reference relative to the monitoring device (e.g. base of the articulated arm).
a local frame of reference R_{L} (fixed) in which a local geometric representation G_{L} of the intermediate element E_{I} is identified (obtained from sectional images which are also identified in the local frame of reference).

FIG. 1 also illustrates the transformations serving to change from one frame of reference to another, namely:
the local transformation ^{L}T_{S} for changing from the monitoring frame of reference R_{S} to the local frame of reference R_{L}. The local transformation ^{L}T_{S} is a constant transformation whatever the position of the imaging device 12.
the preparatory transformation ^{S}T_{I}ⁱ for changing from the image frame of reference R_{I} to the monitoring frame of reference R_{S}. The preparatory transformation ^{S}T_{I}ⁱ is a transformation that varies according to the position of the imaging device 12 during the acquisition of the image considered (the index “i” indicates that the transformation is variable, i being incremented over time).
the image transformation ^{S}T_{I}ⁱ for changing from the image frame of reference R_{I} to the local frame of reference R_{L}. The image transformation ^{L}T_{I}ⁱ is a transformation that varies according to the position of the imaging device 12 during the acquisition of the image considered (the index “i” indicates that the transformation is variable, i being incremented over time).

The transformations are related by the following formula:

^{L}T_{I}ⁱ=^{L}T_{S}·^{S}T_{I}ⁱ

The goal of the present method is to determine the image transformation ^{L}T_{I}ⁱ for registering information on positions (position of the target element) identified in the image frame of reference R_{I} of the image considered to the local frame R_{L}. For this purpose, it is necessary to know the preparatory transformation ^{S}T_{I}ⁱ for the image considered and the local transformation ^{L}T_{S}. The preparatory transformation ^{S}T_{I}ⁱ is suitable for being obtained via the monitoring device 16. Thereby, it remains to determine the local transformation ^{L}T_{S}.

The determination method comprises a preparation phase 100 and an operating phase 200.

The preparatory phase 100 is aimed at determining the local transformation ^{L}T_{S} for changing from the monitoring frame of reference R_{S} to the local frame of reference R_{L}. During the operating phase 200, the determined local transformation ^{L}T_{S} is used to determine the image transformation ^{L}T_{I}ⁱ for determining the position of the target element on the image of interest IM_{I} considered.

The determination method is implemented for an area of the body Z which is immobile in the monitoring frame of reference R_{S}. Thereby, if the area of the body Z moves or is in motion, the different phases of the method have to be reiterated.

The determination method is e.g. implemented by the calculator 18 of the determination device 10, i.e. Is implemented by a computer. More particularly, it is emphasized that the phases and steps described for the determination method relate to an image processing on images of an area of the body Z, acquired beforehand. More particularly, it is emphasized that the present method does not relate to the acquisition of images, the images being only obtained, in the sense of data loading by the calculator 18.

The steps of the method are thus entirely implemented outside the human body, without any contact with the human body. None of the steps is thus of surgical or therapeutic nature. The determination method thus relates to a non-surgical method for determining the position of a target element E_{C} on images of interest of an area of the body Z.

The preparatory phase 100 will now be described in greater detail. More particularly, as indicated hereinabove, the preparatory phase 100 is aimed at identifying the local transformation ^{L}T_{S} (constant) for changing from the monitoring frame of reference R_{S} to the local frame of reference R_{L}.

The preparatory phase 100 comprises a step 110 of obtaining a geometrical representation, called a local geometrical representation G_{L}, of the intermediate element E_{I}. As indicated hereinabove, the term “obtaining” means loading of images by the calculator 18, the images having been previously stored e.g. in a memory of the calculator 18 or coming from a digital data stream to which the calculator 18 is connected.

The local geometric representation G_{L} is a three-dimensional representation. The local geometric representation G_{L} is identified in the local frame of reference R_{L}. The target element E_{C} has a relative position with respect to the intermediate element E_{I} which is known in the local frame of reference R_{L} (e.g. obtained via the sectional images identified in the local frame of reference R_{L} and having served for obtaining the local geometric representation G_{L}).

In an example of embodiment, the local geometric representation G_{L} is obtained depending upon sectional images of the area of the body Z comprising the intermediate element E_{I} and the target element E_{C}. The sectional images were obtained by a medical imaging device. The medical imaging device is e.g. an MRI device, a CBCT device, an optical imaging device, a CT scanner, or an ultrasound device.

The sectional images are e.g. CT scan images IM_{S}. A CT scan image is a slice image of an area of the body Z obtained by positron emission tomography (PET) coupled to a computed tomography (CT), thereby making it possible to image both the intermediate element E_{I} and the target element E_{C}.

More particularly, as illustrated in FIGS. 3, 2D IM_{S} CT scan images are used to reconstruct a 3D representation of the intermediate element E_{I} (iliac arterial system). The image processing software used (e.g. 3D slicer®, see e.g. the following article: Fedorov A, Beichel R, Kalpathy-Cramer J, Finet J, Fillion-Robin J-C, Pujol S, et al. 3D Slicer as an image computing platform for the quantitative Imaging Network. Magn Reson Imaging. Nov 2012; 30(9):1323-41) generates a skeletonization of the arterial system. The skeleton forms the local geometric representation G_{L}.

In the case where the intermediate element E is the iliac arterial system, the skeleton is e.g. formed by curves 2D immersed in the 3D (represented by points or mathematical functions (splines, Bezier curves, etc.)). More particularly, the skeleton materializes the points of the intermediate element E_{I} respecting a projective invariance property (e.g. axis of revolution of the tubes).

The preparatory phase 100 comprises a step 120 of obtaining preparatory images IM_{P} of the area of the body Z. The IM_{P} preparatory images were acquired by the imaging device 12 for different positions of the imaging device 12. Each position of the imaging device 12 corresponds to a different angle of view of the area of the body Z.

The preparatory images IMP each have a known position in the monitoring frame of reference R_{S}. The known position was obtained e.g. according to the position of the imaging device 12 obtained via the monitoring device 16, to which the preparatory transformation ^{S}T_{I}ⁱ is applied, for changing from the image frame of reference R_{I} to the monitoring frame of reference R_{S}. The preparatory transformation ^{S}T_{I}ⁱ is considered known when the position of the imaging device 12 is known. For example, the preparatory transformation ^{S}T_{I}ⁱ according to the position of the imaging device 12 is obtained upstream, e.g. from supplier data or via a prior calibration.

In one example, the preparatory transformation ^{S}T_{I}ⁱ is composed of two elements. A first transformation between the imaging device 12 and the support 14 (at the end of the monitoring device 16, e.g. an instrumented arm). The transformation is fixed and is obtained by a calibration (e.g. with a test pattern). There is then a second transformation between the support 14 (at the end of the monitoring device 16, e.g. an instrumented arm) and the base of the monitoring device 16 (in the present case, the base of the instrumented arm) which is variable due to the movements of the arm. The second transformation is obtained via the monitoring device 16.

The preparatory phase 100 comprises a step 130 of determining the local transformation ^{L}T_{S} for changing from the monitoring frame of reference R_{S} to the local frame of reference R_{L}. The local transformation ^{L}T_{S} is determined according to the local geometric representation G_{L}, to the preparatory images IM_{P}, and to the positions of the imaging device 12 during the acquisition of the preparatory images IM_{P}.

A particular example of determination of the local transformation ^{L}T_{S} is described hereinafter. Nevertheless, the invention applies to any other embodiment serving to determine the local transformation ^{L}T_{S}.

In the example, the determination step 130 comprises a sub-step 130A according to which, for each preparatory image IM_{P}, a geometrical representation of the intermediate element E_{I}, called the annotated representation G_{A}, is determined. In the example, at least three preparatory images IM_{P} are considered.

In the example, each annotated representation G_{A} is a two-dimensional representation. The annotated representations G_{A} are identified in the monitoring frame of reference R_{S} according to the position of the imaging device 12 during the acquisition of said images.

In the example, the annotated representation G_{A} of each preparatory image IM_{P} is obtained by the action of an operator who annotates/highlights/points to a portion of the intermediate element E_{I} on the image (e.g. via a touch screen).

In a variant, the annotated representation G_{A} of each preparatory image IM_{P} is obtained semi-automatically, i.e. by an annotation made by the computer followed by a validation/rectification by an operator. The annotation made by the computer is e.g. performed by a detection algorithm.

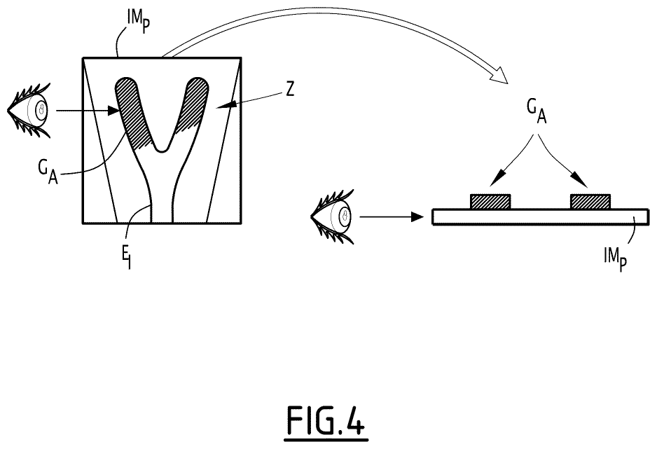

FIG. 4 illustrates an example of a preparatory image IM_{P} on which a part of the intermediate element E_{I} has been identified by annotation, highlighting or pointing (automatically, manually or semi-automatically), the annotations/highlighting/pointing thereby forming the annotated representation G_{A}.

The determination step 130 comprises a sub-step 130B of placement of the local geometric representation G_{L} at a starting position in the monitoring frame of reference R_{S}. The initial position is known (but can be chosen in an arbitrary way).

The determination step 130 comprises a sub-step 130C of determination, in the monitoring frame of reference R_{S}, depending upon the annotated representations G_{A}, of a representation, called the preparatory geometric representation G_{P}.

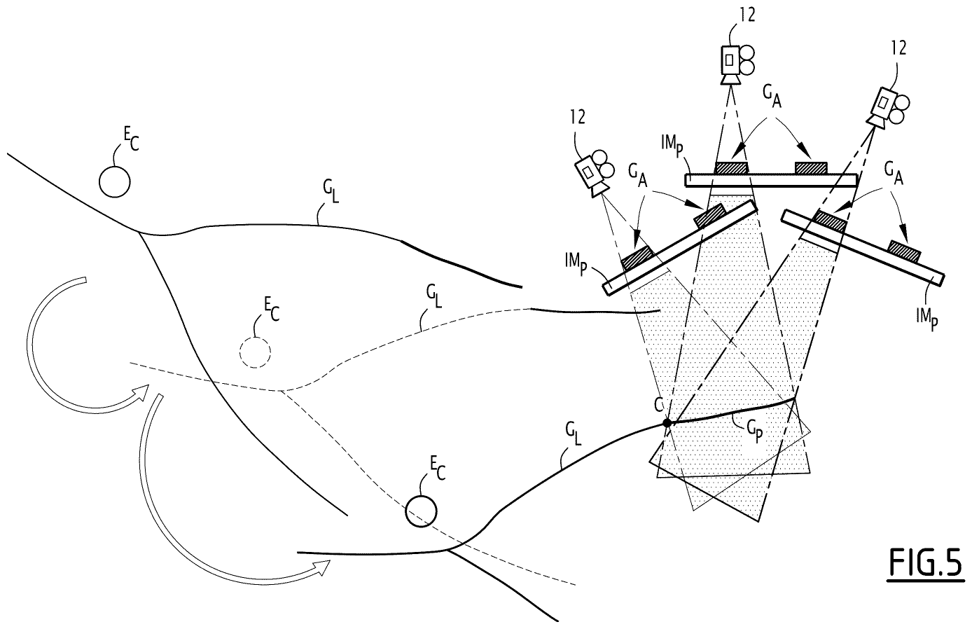

More particularly, in the example illustrated by FIG. 5, the preparatory geometric representation G_{P} is obtained by a projection of the annotated representations G_{A} and an intersection of said projections. Indeed, the intersection of the projective surfaces corresponds to the annotated/highlighted/pointed invariant portion of the intermediate element E_{I} (typically the center C of the artery). The preparatory geometric representation G_{P} is then a “skeleton” of the system at the intersection of the projected surfaces as illustrated in FIG. 5.

The determination step 130 comprises a sub-step 130D of determination of the local transformation ^{L}T_{S} according to the local geometric representation G_{L} and to the preparatory geometric representation G_{P}.

More particularly, the local transformation ^{L}T_{S} is obtained by registering (preferably by superposing) the local geometric representation G_{L} and the preparatory geometric representation G_{P} in the monitoring frame of reference R_{S} (and knowing the initial position of the local geometric representation G_{L} in the monitoring frame of reference R_{S}).

The local transformation ^{L}T_{S} is e.g. obtained by minimizing the distances between the local geometric representation G_{L} and the preparatory geometric representation G_{P} . For example, the minimization consists in minimizing the root mean square (RMS) of the distances between the curves forming the local geometric representation GL and the preparatory geometric representation G_{P}. In another example, the minimization is performed by a so-called gradient descent method or by any other corresponding distance minimization method.

More particularly, FIG. 5 illustrates an example of iterations for minimizing the distances between the local geometric representation G_{L} and the preparatory geometric representation G_{P}.

Preferably, if the degree of similarity is less than a predetermined threshold (threshold set by default in the software but adaptable by the operator), new annotated representations G_{A} are required and the previous steps are repeated with the new annotated representations G_{A}.

Preferably, conditions have been set for the algorithm to stop the computation iterations. The conditions comprise at least one of the following conditions:
the error is sufficiently small (below a predetermined threshold),
the maximum number of iterations is reached, or
the error no longer varies.

The operating phase 200 comprises a step 210 of obtaining an image of interest IM of the area of the body Z. The image of interest MI_{I} was acquired by the imaging device 12 for a position of the imaging device 12 known via the monitoring device 16.

As indicated hereinabove, the term “obtaining” means loading of images by the calculator 18, the images having e.g. been previously stored in a memory of the calculator 18, or coming from a stream of digital data to which the calculator 18 is connected.

The operating phase 200 comprises a step 220 of determination of an image transformation ^{L}T_{I}ⁱ for the image of interest IM_{I} according to the local transformation ^{L}T_{S} determined beforehand and of the position of the imaging device 12 during the acquisition of the image of interest IM_{I}.

More particularly, the position of the imaging device 12 serves to obtain the preparatory transformation ^{S}T_{I}ⁱ for the image of interest IM_{I} considered (e.g. in the same way as in the examples described hereinabove for the preparatory phase). The image transformation ^{S}T_{I}ⁱ for the image of interest MI_{I} is then obtained with the following formula: ^{L}T_{I}ⁱ=^{L}T_{S}·^{S}T_{I}ⁱ.

The operating phase 200 comprises a step 230 of determination of the position PI of the target element E_{C} on the image of interest IM_{I} according to the determined image transformation ^{L}T_{I}ⁱ and of the known relative position of the target element E_{C} with respect to the intermediate element E_{I}.

Preferably, the operating phase 200 comprises a step 240 of display of the image of interest IM_{I} on a screen (of the calculator 18 with a superposed (i.e. augmented reality) representation of the target element E_{C} at the position P_{I} determined on the image of interest IM_{I}.

Preferably, the steps of the operating phase 200 are repeated over time for images of interest IM_{I} corresponding to different positions of the imaging device 12. The repetition is preferably carried out in real time and continuously, i.e. as the images of interest IM_{I} are received by the calculator 18.

Thereby, the present method and the present system 10 serve to determine the position of a target element E_{C} on images of an area of the body Z acquired from different viewpoints. For this purpose, an intermediate element E_{I} is used to register a representation of the target element E_{C} on the image of interest IM_{I} considered.

One of the advantages of the present method is that once the local transformation ^{L}T_{S} has been obtained, and provided that the area of the body Z remains immobile, the registration can be performed on images of interest IM_{I} of the area of the body Z acquired regardless of the position of the imaging device 12. In other words, the imaging device 12 can be freely moved to acquire different images of interest IM_{I} of the area of the body Z without having to again perform a preparatory calibration phase.

The method can thus be used to locate invisible sentinel lymph nodes (i.e. target element E_{C}) by the imaging device 12 (e.g. endoscopic camera, laparoscope). Indeed, the intermediate element E_{I} (iliac arterial system) serves to register previously obtained information (preferably coming from PET images coupled with a preoperative CT scan (computed tomography) on which the lymph nodes would have been identified beforehand) on the images acquired by the imaging device 12. It is thereby possible to generate a visible superposition in augmented reality.

A person skilled in the art will understand that the embodiments and variants described above can be combined so as to form new embodiments provided that same are technically compatible.

For example, although the present description has given an example with only one intermediate element and only one target element, a person skilled in the art would understand that the invention also applies to the case where a plurality of intermediate elements and/or a plurality of target elements are considered.

In addition, it should be noted that in the examples of the present description, the transformations between the different frames of reference have been described along the direction of the arrows in FIG. 1. Nevertheless, a person skilled in the art would understand that the present description also applies in the case where:
the local transformation considered serves to change from the local frame of reference R_{L} to the monitoring frame of reference R_{S} (transformation ^{S}T_{L}), and
the preparatory transformation considered serves to change from the monitoring frame of reference R_{S} to the image frame of reference R_{I} (transformation ^{I}T_{S}ⁱ), and
the image transformation considered makes it possible to change from the local frame of reference R_{L} to the image frame of reference R_{I} (transformation ^{I}T_{L}ⁱ).

In such case, the transformations are related by the following formula:

^{I}T_{L}ⁱ=^{I}T_{S}ⁱ·^{S}T_{L}

1. A method for determining the position of at least one target element on at least one image of interest of an area of the body, the area of the body comprising at least one intermediate element imaged on the image of interest, the image of interest having been acquired by an imaging device, each image acquired by the imaging device having a specific frame of reference, called the image frame of reference, in which elements of image are identified, the position of the imaging device at each acquisition of images being identified in a fixed frame of reference, called the monitoring frame of reference, the area of the body being immobile in the monitoring frame of reference during the implementation of the method, the method being implemented by a computer and comprising:
a. a preparation phase comprising the following steps:
i. obtaining a geometrical representation, called the local geometrical representation, of the intermediate element, the local geometrical representation being identified in a fixed geometrical frame of reference, called the local frame of reference, the target element having a relative position with respect to the intermediate element which is known in the local frame of reference,
ii. obtaining preparatory images of the area of the body, the preparatory images having been acquired by the imaging device for different positions of the imaging device, each position of the imaging device corresponding to a different angle of view of the area of the body,
iii. determination of a transformation, known as the local transformation, between the monitoring frame of reference and the local frame of reference according to the local geometric representation, the preparatory images, and the positions of the imaging device during the acquisition of the preparatory images,
b. an operating phase comprising the following steps:
i. obtaining an image of interest of the area of the body,
ii. determination of a transformation, called the image transformation between the local frame of reference and the image frame of reference of the image of interest, the image transformation being determined according to the determined local transformation and to the position of the imaging device during the acquisition of the image of interest, and
iii. determination of the position of the target element on the image of interest according to the determined image transformation and the known relative position of the target element with respect to the intermediate element.

2. The method according to claim 1, wherein the steps of the operating phase are repeated over time for images of interest corresponding to different positions of the imaging device.

3. The method according to claim 1, wherein the operating phase comprises a step of displaying the image of interest on a screen with a superimposed representation of the target element at the position determined on the image of interest.

4. The method according to claim 1, wherein the local geometric representation is obtained depending upon sectional images of the area of the body comprising the intermediate element and the target element, the sectional images having been acquired by a medical imaging device.

5. The method according to claim 1, wherein the step of determination of the local transformation comprises:
a. the determination, for each preparatory image, of a geometrical representation, called the annotated representation, of the intermediate element, the annotated representations being identified in the monitoring frame of reference according to the position of the imaging device during the acquisition of said images,
b. the placement of the local geometric representation at an initial position in the monitoring frame of reference,
c. the determination, in the monitoring frame, according to the annotated representations, of a representation, called the preparatory geometric representation, and
d. the determination of the local transformation by registration of the local geometric representation and of the preparatory geometric representation in the monitoring frame of reference.

6. The method according to claim 5, wherein the preparatory geometric representation is obtained by a projection of the annotated representations and an intersection of said projections.

7. The method according to claim 1, wherein the intermediate element has an invariant shape in projective geometry.

8. The method according to claim 1, wherein the intermediate element is an arterial system, and the target element is a lymph node.

9. A system for determining the position of at least one target element on at least one image of interest of an area of the body, the area of the body comprising one intermediate element imaged on the image of interest, the image of interest having been acquired by an imaging device, each image acquired by the imaging device having a specific frame of reference, called the image frame of reference, in which elements of image are identified, the position of the imaging device at each acquisition of images being identified in a fixed frame of reference, called the monitoring frame of reference the system comprising a calculator configured to implement the steps of a method according to claim 1.

10. The system according to claim 11, wherein the system further comprises:
a. the imaging device for the acquisition of preparatory images and of images of interest,
b. a support on which the imaging device is mounted, and
c. a monitoring device connected to the support for moving the imaging device and monitoring the movements of the imaging device.

11. The method according to claim 8, wherein the arterial system is the iliac arterial system.

12. The method according to claim 8, wherein the lymph node is a sentinel lymph node.
